(19) United States
(12) Patent Application Publication
(10) Pub. No.: US 2023/0380751 A1
(43) Pub. Date: Nov. 30, 2023

(54) Brain Impact Sensor and Methods of Using the Same

(71) Applicant: Thomas Pizur, Wauconda, IL (US)

(72) Inventor: Thomas Pizur, Wauconda, IL (US)

(21) Appl. No.: 17/824,435

(22) Filed: May 25, 2022

(51) Int. Cl. A61B 5/00; A61B 5/11

(52) U.S. Cl. CPC A61B 5/4088; A61B 5/6803; A61B 5/112; A61B 5/4064; A61B 5/746; A61B 5/002; A61B 2562/0219

(57) ABSTRACT

A brain impact sensor apparatus comprises a sensor attached to a processor/controller that is held close to the top of a user's head. The sensor records vibrations caused by movement of the user. Specifically, as a user walks, runs, or otherwise carries on daily activities, the impacts of the user's gait is recorded in the sensor apparatus held close to the top of the user's head. Thus, the sensor and processor/controller are configured to record repetitive vibrations caused by the user's gait. Analysis of the recorded vibrations may aid in diagnosing disease or may aid in recovery via physical therapy. Methods of using the same are further provided.

### CROSS-REFERENCE TO RELATED APPLICATIONS

The present invention claims priority to U.S. Provisional Pat. App. No. 63/209,811, titled “Brain Impact Sensor and Methods of Using the Same,” filed Jun. 11, 2021, which is incorporated herein by reference in its entirety.

### TECHNICAL FIELD

The present invention relates to a brain impact sensor apparatus. Specifically, the present invention comprises a sensor attached to a processor/controller that is held close to the top or crown of a user's head. The sensor records vibrations caused by movement of the user. Specifically, as a user walks, runs, or otherwise carries on daily activities, the impacts of the user's gait is recorded in the sensor apparatus held close to the top of the user's head. Thus, the sensor and processor/controller are configured to record repetitive vibrations caused by the user's gait. Analysis of the recorded vibrations may aid in diagnosing disease or may aid in recovery via physical therapy. Methods of using the same are further provided.

### BACKGROUND

It is generally well-known that individuals that suffer from repeated concussive blows to the head are often diagnosed with traumatic brain injuries that can lead to long-term neurological problems, including memory problems, speech problems, and dementia. Often, these concussive blows to the head can be received during play, such as in sports or the like. For example, boxers and football players often report receiving concussions that cause cumulative damage to their brains.

What is less well-known is what effect repeated lesser vibrations can have on human brains. Specifically, as a person walks or runs, he or she strikes the ground with his or her foot, sending vibrations through the person's body. It is theorized that over time, these lesser vibrations may have long term effects on a person's brain health. For example, repeated concussive impacts on a person's brain may affect the person's sleep patterns, which may further affect their cognitive abilities. A person can adjust their gait when running or walking to minimize these vibrations and, therefore, minimize damage to the brain.

A person's walking style may affect his or her cognitive abilities, based on increased or decreased repetitive impact of his or her feet on ground surfaces that can translate to his or her brain. Typically, a person's walking style is learned from his or her parents. Also, a person's body style may affect his or her gait. Most of the time, people walk without thinking about it, and it is possible that simply how a person walks can affect their long-term cognitive abilities.

Moreover, recent changes in environmental factors can increase the number of repetitive impacts on a person's brain as he or she walks or runs. Specifically, over the last 100 years or so, humans have relied more and more on concrete and other hard paved surfaces. For much of the evolution of modern humans, human beings did not walk on concrete. Further, many humans are under large amounts of stress, and this stress can translate to increased vibrations. For example, stressed individuals tend can be relatively aggressive with their movements, which can affect their gait. Many stressed individuals can stomp their feet or make other exaggerated movements that can translate through the body to the brain.

Moreover, modern shoe design may affect the repetitive impacts. Specifically, shoes often are not able to flex properly to absorb and therefore transfer a large amount of the repetitive impact. In addition, it is common for humans to be overweight, and the added weight may cause the repetitive impacts to have a larger effect. Moreover, humans simply live longer, and the amount of cumulative repetitive impacts on a person's body and, more specifically, his or her brain, may simply be larger. Finally, modern humans spend much time sitting or otherwise sedentary, which may lead to bad habits while walking.

It is often difficult to measure how much impact a person's gait may have on a person's brain. If a person could determine how his or her walk may affect long-term brain trauma, he or she may be able to adjust their gait to minimize such impacts. A need, therefore, exists for a brain impact sensor apparatus that records vibrations that may be transferred from a person's feet to his or her brain. Specifically, a need exists for a brain impact sensor apparatus that provides a readily viewable read-out of vibrations recorded by the brain impact sensor as the user uses the brain impact sensor to measure the impact of vibrations on his or her gait. More specifically, a need exists for a brain impact sensor apparatus that transfers such results to a viewable screen of a computing device, such as a smart phone screen, a computer screen, or the like for viewing of the same.

Normally, a person without injury or illness should walk with a balanced gait, with one side of his or her body not bearing more or less weight than the other side. However, injury or illness may affect a person's gait. Specifically, an injury to a person's feet, legs, knees, hips, back or other like body parts may cause a person to walk unbalanced or with a limp. Moreover, an illness may affect a person's bones, ligaments, tendons, musculature, internal organs, or the like, and may cause a person to walk with an unbalanced gait. Correction of a person's gait may be done via the watchful care of a physical therapist, a doctor, or other medical professional. However, without a well-trained eye, it may be difficult for a person to know when he or she is walking with an unbalanced gait. A need, therefore, exists for a sensor apparatus that measures a user's gait. Specifically, a need exists for a sensor apparatus that measures how balanced or unbalanced a user's gait may be.

Slight adjustments in a person's gait may help the person overcome the injury or illness. It may be difficult for a person to obtain the review by a medical professional necessary to make corrections, especially when he or she is unaware or cannot see what needs to change or how to do it. A need, therefore, exists for a sensor apparatus that allows a user to adjust his or her gait. Specifically, a need exists for a sensor apparatus that provides immediate feedback to a user thereof of whether his or her gait is problematic, and how to fix the same.

A person's gait may also identify certain types of dementia. There are different types of dementia, and some types may affect how a person walks. For example, it is known that individuals with Lewy body dementia often have associated physical movement problems, such as slow, stiff movements or problems with balance. Oftentimes, treatments for Lewy body dementia involve some more of physiotherapy. In general, the gaits of people with Lewy body dementia are distinguishable from people with other types of dementia, as those individuals had unique walking patterns; their steps can be more variable and asymmetric. However, oftentimes these differences in gait are subtle and are not noticeable to many in the medical field. Thus, a recognition of the physical limitations associated with certain types of dementia may be useful in correct diagnoses and can lead to better treatment. A need, therefore, exists for a sensor apparatus that may be utilized to detect certain illnesses. Specifically, a need exists for a sensor apparatus that may be utilized to aid in diagnosing certain types of dementia or other like diseases.

### SUMMARY OF THE INVENTION

The present invention relates to a brain impact sensor apparatus. Specifically, the present invention comprises a sensor attached to a processor/controller that is held close to the top or crown a user's head. The sensor records vibrations caused by movement of the user. Specifically, as a user walks, runs, or otherwise carries on daily activities, the impacts of the user's gait is recorded in the sensor apparatus held close to the top of the user's head. Thus, the sensor and processor/controller are configured to record repetitive vibrations caused by the user's gait. Analysis of the recorded vibrations may aid in diagnosing disease or may aid in recovery via physical therapy. Methods of using the same are further provided.

To this end, in an embodiment of the present invention, a brain impact sensor apparatus is provided. The brain impact sensor apparatus comprises: a sensor configured to be positioned at a crown of a user's head; and a processor electronically connected to the sensor, wherein the sensor is configured to measure low-level impacts to the user's body during use thereof.

In an embodiment, the sensor is disposed in a hat.

In an embodiment, the sensor is place at the crown of the hat.

In an embodiment, is disposed in a head band.

In an embodiment, the sensor, when positioned at a crown of a user's head, contacts the crown of the user's head.

In an embodiment, the sensor comprises an accelerometer.

In an embodiment, the brain impact sensor apparatus further comprises a communication module configured to communicate with a separate computing device.

In an embodiment, the communication module is configured to communicate via a wired or a wireless connection to the separate computing device.

In an embodiment, the separate computing device is selected from the group of a personal computer, a smart phone, a tablet computer, and a smart watch.

In an embodiment, the sensor is configured to measure the low-level impacts to the user's body while the user is walking or running.

In an alternate embodiment of the present invention, a method of using a brain impact sensor apparatus is provided. The method comprises the steps of: providing a brain impact sensor apparatus comprising a sensor configured to be positioned at a crown of a user's head, and a processor electronically connected to the sensor, wherein the sensor is configured to measure low level impacts to a user's body during use thereof; measuring low-level impacts to the user's body to form impact data; storing the impact data within a storage module within the brain impact sensor apparatus; and sending the impact data to a separate computing device.

In an embodiment, the sensor is disposed in a hat or a headband worn on the user's head.

In an embodiment, the low-level impacts to the user's body are caused by the user walking or running.

In an embodiment, the impact data is processed at the processor electronically connected to the sensor to form processed impact data.

In an embodiment, the impact data is processed by the computing device to form processed impact data.

In an embodiment, the method further comprises the step of: providing a communication module configured to communicate with the separate computing device; and sending the impact data to the separate computing device via the communication module.

In an embodiment, the method further comprises the step of: sending the impact data to the separate computing device via a wired or wireless connection between the brain impact sensor apparatus and the separate computing device.

In an embodiment, the separate computing device is selected from the group of a personal computer, a smart phone, a tablet computer, and a smart watch.

In an embodiment, the method further comprises the step of: analyzing the impact data at the separate computing device.

In an embodiment, the method further comprising the step of: providing an audible or visual alarm; analyzing the impact data; and triggering the alarm after analyzing the impact data.

It is, therefore, an advantage and objective of the present invention to provide a brain impact sensor apparatus that records vibrations that may be transferred from a person's feet to his or her brain.

Specifically, it is an advantage and objective of the present invention to provide a brain impact sensor apparatus that provides a readily viewable read-out of vibrations recorded by the brain impact sensor as the user uses the brain impact sensor to measure the impact of vibrations on his or her gait.

More specifically, it is an advantage and objective of the present invention to provide a brain impact sensor apparatus that transfers such results to a viewable screen, such as a smart phone screen or the like for viewing of the same.

Moreover, it is an advantage and objective of the present invention to provide a sensor apparatus that measures a user's gait.

Specifically, it is an advantage and objective of the present invention to provide a sensor apparatus that measures how balanced or unbalanced a user's gait may be.

In addition, it is an advantage and objective of the present invention to provide a sensor apparatus that allows a user to adjust his or her gait.

Specifically, it is an advantage and objective of the present invention to provide a sensor apparatus that provides immediate feedback to a user thereof of whether his or her gait is problematic, and how to fix the same.

Further, it is an advantage and objective of the present invention to provide a sensor apparatus that may be utilized to detect certain illnesses.

Still further, it is an advantage and objective of the present invention to provide a sensor apparatus that may be utilized to aid in diagnosing certain types of dementia or other like diseases.

Additional features and advantages of the present invention are described in, and will be apparent from, the detailed description of the presently preferred embodiments and from the drawings.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates a user having a hat incorporating a brain impact sensor apparatus therein configured to measure low level impacts of the user's body during use thereof, in an embodiment of the present invention.

FIG. 2 illustrates a close-up view of a user and a hat comprising the brain impact sensor apparatus, including the brain impact sensor and processor.

### DETAILED DESCRIPTION OF THE PRESENTLY PREFERRED EMBODIMENTS

The present invention relates to a brain impact sensor apparatus. Specifically, the present invention comprises a sensor attached to a processor/controller that is held close to a user's head. The sensor records vibrations caused by movement of the user. Specifically, as a user walks, runs, or otherwise carries on daily activities, the impacts of the user's gait is recorded in the sensor apparatus held close to the top of the user's head. Thus, the sensor and processor/controller are configured to record repetitive vibrations caused by the user's gait. Analysis of the recorded vibrations may aid in diagnosing disease or may aid in recovery via physical therapy. Methods of using the same are further provided.

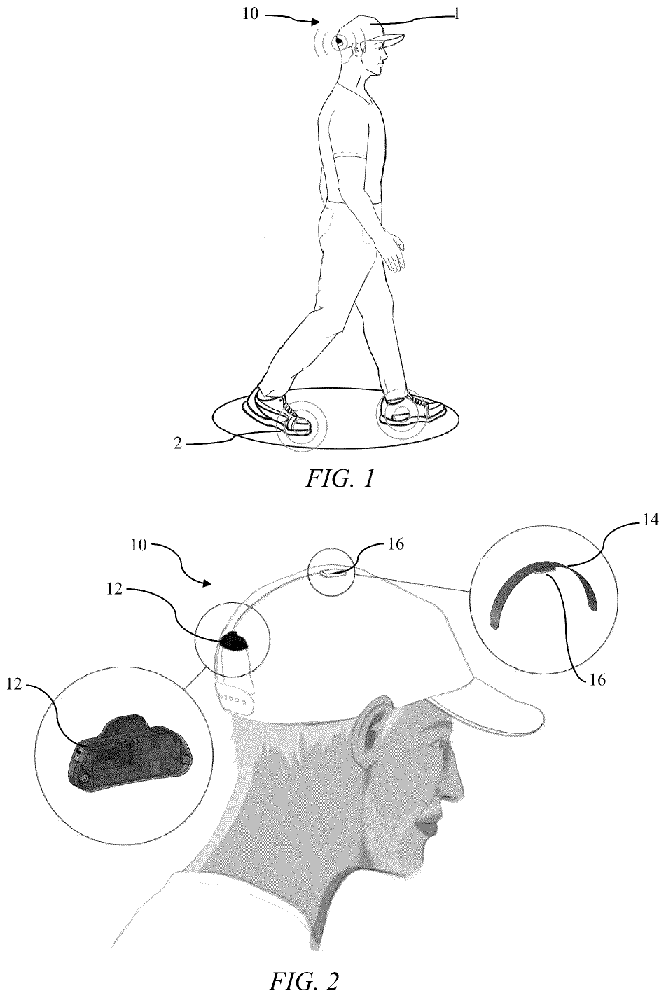

Now referring to the drawings, FIG. 1 illustrates a user wearing a hat 1 comprising a brain impact sensor apparatus 10 therein for measuring low-level impacts 2 when walking, running, or otherwise moving. FIG. 2 illustrates a brain impact sensor apparatus 10 in an embodiment of the present invention. The brain impact sensor apparatus 10 comprises a base housing 12, an accelerometer housing 14 extending from and electronically connected to the base housing 12, and an accelerometer 16. Within the base housing 12 may be components useful for processing, controlling, measuring and/or recording movements and vibrations detected by the accelerometer 16. Specifically, the base housing 12 may comprise a processor for detecting and measuring movement of the accelerometer 16 and a communication means, such as a Bluetooth module for communicating to an external computing device, such as, for example, a mobile device, such as a smart phone, a computer, or other like computing device. Preferably, the processor communicates with an external computing device and transmits data recorded and/or processed by the processor from the accelerometer 16.

An exemplary processor may be an Arduino mini-Pro Atmega 328P microcontroller. The communication means may be an Arduino HC-05 Bluetooth Module. The accelerometer may be an Adafruit LIS3DH Triple-Axis Accelerometer. However, each of these components may be any other module or device that accomplishes the same function and purpose as described herein, and the present invention should not be limited as described. Moreover, the base housing may further comprise a battery and a battery charger, preferably a 3.7 V Lithium-Ion Battery with a Lithium-Ion Battery Charger, for powering the electronic components described herein, namely the microcontroller, the Bluetooth module, and the accelerometer 16.

The apparatus 10 may be positioned within headgear, such as a head band or the hat 1, as illustrated in FIGS. 1 and 2. In a preferred embodiment, the apparatus 10 may be disposed within the hat 1 such that the accelerometer 16 is positioned at the highest point of a user's head, specifically, the crown of the user's head, as illustrated in FIG. 2. The accelerometer housing 14 may comprise an arching band that may hold the accelerometer 16 in position on the top or crown of the user's head; specifically, within the hat 1. The processor 12 may be positioned on a rear of the hat 1, such as within the space of the hat 1 where the hat 1 may be adjusted, as shown in FIG. 2. The processor 12 may be electronically connected to the accelerometer 16 via one or more cables or wires, thereby receiving the sensor data from the accelerometer 16, which may then be passed onto a computing device (not shown) via wireless communication protocol, such as Bluetooth or the like.

For example, the hat 1 may be a baseball hat, and may include sufficient space and adjustability to hold the components thereof inside for concealing the same and for comfort of the user. Positioning the accelerometer sensor 16 at or near the top or crown of the user's head via the housing 14 may provide the most accurate data, and thus is preferred. More specifically, the accelerometer 16 may be housed at the crown of the skull and aligned such that the proper axis is in the upright position and at the highest point of the individual.

When in use, a user wears the hat 1 containing the apparatus 10 disposed therein. As he or she walks, runs, or otherwise carries on his or her normal day, movements and vibrations detected by the accelerometer measured, recorded and/or processed by the processor 12. The accelerometer 16 may, for example, measure the intensity of energy generated by the user's heels striking the ground and transferred to his or her brain. Thus, the user's gait, size of impact as the user walked, and other like impact data may be recorded, and analyzing the same may allow a viewer to determine whether the user is causing micro-concussions to impact his or her brain. Moreover, analysis of the extent of impact caused by the left foot and/or the right foot may be used to determine whether the user is walking in a balanced manner. If not, then the user may view differences in the energy intensities of right foot versus left foot walking and may adjust accordingly to achieve a balanced walking gait. In addition, certain patterns may be detected within a user's gait that may be utilized for diagnoses of diseases and/or conditions.

The data generated by the accelerometer 16 and recorded by the processor may be further processed for viewing using the processor, and communicated wirelessly to an external computing device, such as a smart phone, for viewing and further analysis. Thus, impacts detected by the apparatus 10 may be able to track daily impacts arising from mere daily walking. Thus, a user may view the data recorded by the apparatus 10 on his or her smart phone and may adjust his or her gait to reduce or eliminate dangerous micro-concussions caused by the user's gait. Moreover, after analysis, either at the processor or on a user's smart phone or other computing device, an alarm, warning, or other notification may be relayed to the user, either by sight, sound, vibration, or the like, providing a notification to the user that the user is walking or otherwise moving in a way that he or she should not. The warning may be provided to give instant feedback to the user to make a change to his or her gait or other movement, thereby minimizing stress impacts to the user's brain, in real-time. For example, if a user is stressed and translated that stress to stomps or other exaggerated movements, the sensor may detect such movements and provide an immediate indication to the user. Once recognized, the user may be able to control his or her movements, and decrease or eliminate the stomps or other movements, and decrease the stressor.

Thus, a user may be provided with cumulative reports and analysis and/or real-time feedback that his or her gait and/or other movements is causing too much energy transfer to the user's brain and may be instantly provided with details that may cause the user to change his or her gait and/or other movements. This may lead to decreased cumulative impacts over time, leading to a healthier brain, better sleep, decreased incidences of disease and mental health issues, and the like.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A brain impact sensor apparatus comprising:
a sensor configured to be positioned at a crown of a user's head; and
a processor electronically connected to the sensor,
wherein the sensor is configured to measure low-level impacts to the user's body during use thereof.

2. The brain impact sensor apparatus of claim 1 wherein the sensor is disposed in a hat.

3. The brain impact sensor apparatus of claim 2 wherein the sensor is place at the crown of the hat.

4. The brain impact sensor apparatus of claim 1 wherein the sensor is disposed in a head band.

5. The brain impact sensor apparatus of claim 1 wherein the sensor, when positioned at a crown of a user's head, contacts the crown of the user's head.

6. The brain impact sensor apparatus of claim 1 wherein the sensor comprises an accelerometer.

7. The brain impact sensor apparatus of claim 1 further comprising:
a communication module configured to communicate with a separate computing device.

8. The brain impact sensor of claim 7 wherein the communication module is configured to communicate via a wired or a wireless connection to the separate computing device.

9. The brain impact sensor of claim 7 wherein the separate computing device is selected from the group of a personal computer, a smart phone, a tablet computer, and a smart watch.

10. The brain impact sensor apparatus of claim 1 wherein the sensor is configured to measure the low-level impacts to the user's body while the user is walking or running.

11. A method of using a brain impact sensor apparatus comprising the steps of:
providing a brain impact sensor apparatus comprising a sensor configured to be positioned at a crown of a user's head, and a processor electronically connected to the sensor, wherein the sensor is configured to measure low level impacts to a user's body during use thereof;
measuring low-level impacts to the user's body to form impact data;
storing the impact data within a storage module within the brain impact sensor apparatus; and
sending the impact data to a separate computing device.

12. The method of claim 11 wherein the sensor is disposed in a hat or a headband worn on the user's head.

13. The method of claim 11 wherein the low-level impacts to the user's body are caused by the user walking or running.

14. The method of claim 11 wherein the impact data is processed at the processor electronically connected to the sensor to form processed impact data.

15. The method of claim 11 wherein the impact data is processed by the computing device to form processed impact data.

16. The method of claim 11 further comprising the steps of:
providing a communication module configured to communicate with the separate computing device; and
sending the impact data to the separate computing device via the communication module.

17. The method of claim 11 further comprising the step of:
sending the impact data to the separate computing device via a wired or wireless connection between the brain impact sensor apparatus and the separate computing device.

18. The method of claim 11 wherein the separate computing device is selected from the group of a personal computer, a smart phone, a tablet computer, and a smart watch.

19. The method of claim 11 further comprising the step of:
analyzing the impact data at the separate computing device.

20. The method of claim 11 further comprising the step of:
providing an audible or visual alarm;
analyzing the impact data; and
triggering the alarm after analyzing the impact data.
